(19) United States
(12) Patent Application Publication
(10) Pub. No.: US 2021/0361260 A1
(43) Pub. Date: Nov. 25, 2021

(54) ULTRASONIC TRANSDUCER ARRAY HAVING VARYING CAVITY DIAMETER PROFILE

(71) Applicants: Lingyun Miao, Fremont, CA (US); Sarp Satir, San Francisco, CA (US)

(72) Inventors: Lingyun Miao, Fremont, CA (US); Sarp Satir, San Francisco, CA (US)

(73) Assignee: Butterfly Network, Inc., Guilford, CT (US)

(21) Appl. No.: 17/326,938

(22) Filed: May 21, 2021

Related U.S. Application Data
(60) Provisional application No. 63/029,010, filed on May 22, 2020.

(51) Int. Cl. A61B 8/00; B06B 1/02; A61B 8/08

(52) U.S. Cl. CPC A61B 8/4494; A61B 8/5207; B06B 1/0292

(57) ABSTRACT

An ultrasonic transducer array includes a plurality of functional micromachined ultrasonic transducers (MUTs), each having a cavity of a first diameter. One or more groups of non-functional MUTs are disposed about a perimeter of the functional MUTs, the one or more groups of non-functional MUTs having a cavity of a second diameter that is smaller than the first diameter.

### CROSS-REFERENCE TO RELATED APPLICATIONS

This application claims the benefit of U.S. Provisional Application Ser. No. 63/029,010, filed on May 22, 2020, under Attorney Docket No. B1348.70187US00 and entitled “ULTRASONIC TRANSDUCER ARRAY HAVING VARYING CAVITY DIAMETER PROFILE,” which is hereby incorporated herein by reference in its entirety.

### BACKGROUND

The present disclosure relates generally to micromachined ultrasonic transducers and, more specifically, to an ultrasonic transducer array having a varying cavity diameter profile such as a radially outward tapered cavity diameter profile.

Ultrasound devices may be used to perform diagnostic imaging and/or treatment, using sound waves with frequencies that are higher than those audible to humans. When pulses of ultrasound are transmitted into tissue, sound waves are reflected off the tissue with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound images.

Some ultrasound imaging devices may be fabricated using micromachined ultrasonic transducers, including a flexible membrane suspended above a substrate. A cavity is located between part of the substrate and the membrane, such that the combination of the substrate, cavity and membrane form a variable capacitor. When actuated by an appropriate electrical signal, the membrane generates an ultrasound signal by vibration. In response to receiving an ultrasound signal, the membrane is caused to vibrate and, as a result, generates an output electrical signal.

### SUMMARY

In one aspect, an ultrasonic transducer array includes a plurality of functional micromachined ultrasonic transducers (MUTs) each having a cavity of a first diameter. One or more groups of non-functional MUTs are disposed about a perimeter of the functional MUTs, the one or more groups of non-functional MUTs having a cavity of a second diameter that is smaller than the first diameter.

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 is a cross-sectional view of an exemplary micromachined ultrasonic transducer.

FIG. 2 schematically illustrates an infinite 2D transducer array grid.

FIG. 3 schematically illustrates a corner of a finite 2D transducer array.

FIG. 4 illustrates the array shown in FIG. 3, using dummy transducers at the array perimeter.

FIG. 5 illustrates an ultrasonic transducer array having a tapered cavity diameter profile, in accordance with an embodiment.

FIG. 6 illustrates an ultrasonic transducer array having a tapered cavity diameter profile, in accordance with an embodiment.

FIG. 7 illustrates an ultrasonic transducer array having electrical connections to ground and integrated circuitry, in accordance with an embodiment.

FIG. 8 is a cross-sectional view of an example of a micromachined ultrasonic transducer and a dummy transducer, in accordance with an embodiment.

### DETAILED DESCRIPTION

The techniques and structures described herein relate to micromachined ultrasonic transducers (MUTs) having enhanced reliability. In one aspect, an ultrasonic transducer array design includes a plurality of functional MUTs, each having a cavity of a first diameter. One or more groups of non-functional MUTs are disposed about a perimeter of the functional MUTs, the one or more groups of non-functional MUTs having a cavity of a second diameter that is smaller than the first diameter. In another aspect, at least three groups of non-functional MUTs have successively smaller cavity diameters in the outward direction with respect to the array. This in turn may alleviate or resolve a transducer array edge effect issue that can occur in MUT arrays. For example, by forming groups of non-functional MUTs having tapered cavity diameters at the transducer array edges, improved acoustic performance may be achieved in comparison to other approaches such as having non-functional perimeter MUTs with the same diameter as the active transducers. Non-functional MUTs may have structurally similar cavities as functional MUTs, but are rendered non-functional or inactive for imaging purposes, such as (for example) by omitting the formation of functional electrodes or by permanently electrically connecting the electrodes of the non-functional MUTs to ground (e.g., by a bypass line). Engineered tapered cavities at the transducer array edges may not only enhance imaging quality by reducing the reflection of acoustic waves at the array boundary, but also can increase chip area usage by reducing the overall inactive or “dummy” transducer area within the entire transducer array.

One type of transducer suitable for use in ultrasound imaging devices is a MUT, which can be fabricated from, for example, silicon and configured to transmit and receive ultrasound energy. MUTs may include capacitive micromachined ultrasonic transducers (CMUTs) and piezoelectric micromachined ultrasonic transducers (PMUTs), both of which can offer several advantages over more conventional transducer designs such as, for example, lower manufacturing costs and fabrication times and/or increased frequency bandwidth. With respect to the CMUT, the basic structure is a parallel plate capacitor with a rigid bottom electrode and a top electrode residing on or within a flexible membrane. Thus, a cavity is defined between the bottom and top electrodes. In some designs (such as those produced by the assignee of the present application for example), a CMUT may be directly integrated on an integrated circuit that controls the operation of the transducer. One way of manufacturing a CMUT is to bond a membrane substrate to an integrated circuit substrate, such as a complementary metal oxide semiconductor (CMOS) substrate. This may be performed at temperatures sufficiently low to prevent damage to the integrated circuit.

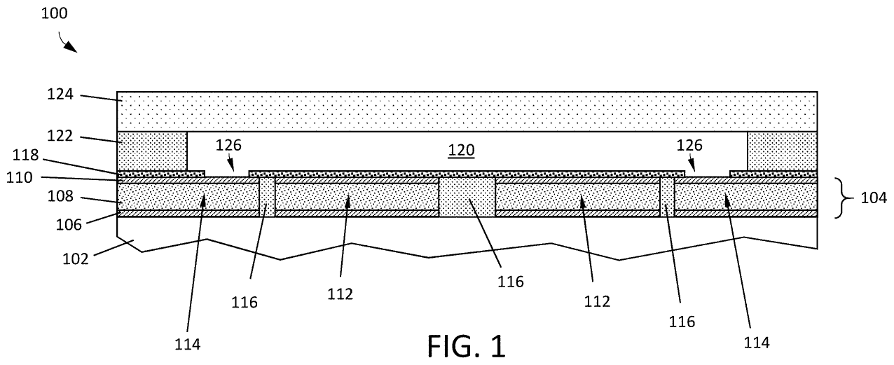

Referring initially to FIG. 1, there is shown a cross-sectional view of an exemplary micromachined ultrasonic transducer 100, such as a CMUT. The transducer 100 includes a substrate, generally designated by 102, (e.g., a CMOS substrate, such as silicon) having one or more layers, such as for example: CMOS integrated circuits and wiring layers, one more insulation/passivation layers, and one or more wiring redistribution layers. A transducer bottom electrode layer, designated generally at 104, is disposed over the substrate 102 and includes patterned regions of a metal layer stack. In the example depicted, the metal layer stack may include a first layer 106 of titanium nitride (TiN), a layer 108 of titanium (Ti) and a second layer 110 of TiN. The transducer bottom electrode layer 104 is patterned in a manner so as to define structures such as, for example, a transducer bottom electrode 112 (e.g., in a “donut” or ring configuration) and bypass metal structures 114, between which are located regions of an insulation layer 116 (e.g., silicon oxide (SiO₂)). It should be appreciated, however, that since specific substrate and transducer bottom electrode patterns are not the focus of the present disclosure, other such patterns are also contemplated. It should further be understood that electrical connections (e.g., such as vias) to the bottom electrode layer structures are not the focus of the present disclosure and, as such, are included within the general substrate region 102 of FIG. 1.

Still referring to FIG. 1, a bottom cavity layer 118 is disposed over the transducer bottom electrode layer 104. The bottom cavity layer 118 may include, for example, an electrically insulating, thin film layer stack including an SiO₂ layer deposited by chemical vapor deposition (CVD) and an aluminum oxide (Al₂O₃) layer deposited by atomic layer deposition (ALD). A transducer cavity 120 is defined by lithographic patterning and etching of a membrane support layer 122 that is formed on the bottom cavity layer 118. The membrane support layer 122 may be an insulating layer, such as SiO₂ for example, the remaining portions of which provide a support surface to which a flexible transducer membrane 124 (e.g., highly doped silicon at a dopant concentration of about 1×10¹⁸ atoms/cm³ to about 1×10¹⁹ atoms/cm³) is bonded. In order to preserve the integrity and functionality of the various CMOS devices residing within the substrate 102 (such as CMOS circuits and wiring layers at or below transducer bottom electrode layer 104), a relatively low temperature bonding process (e.g., less than about 450° C.) is employed for bonding the transducer membrane 124 to the membrane support layer 122.

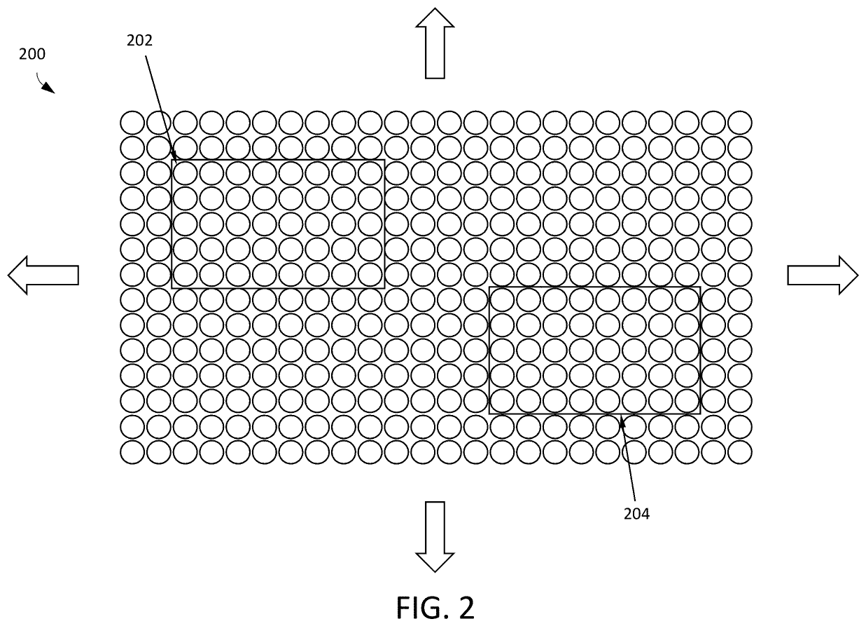

A two-dimensional (2D) transducer array grid of CMUTs may be used for acoustic imaging, with a CMUT cavity disposed at each intersection of the grid. FIG. 2 schematically illustrates an infinite 2D transducer grid 200. In this “ideal” case, one can make use of (for example) region 202 or region 204 as the imaging field (array aperture) without any difference between them since the acoustic boundary conditions are the same for both regions. However, in a real ultrasound device, there are transducer array edge effects to deal with as the boundary conditions change at the array edges (perimeter) as compared to the ideal case.

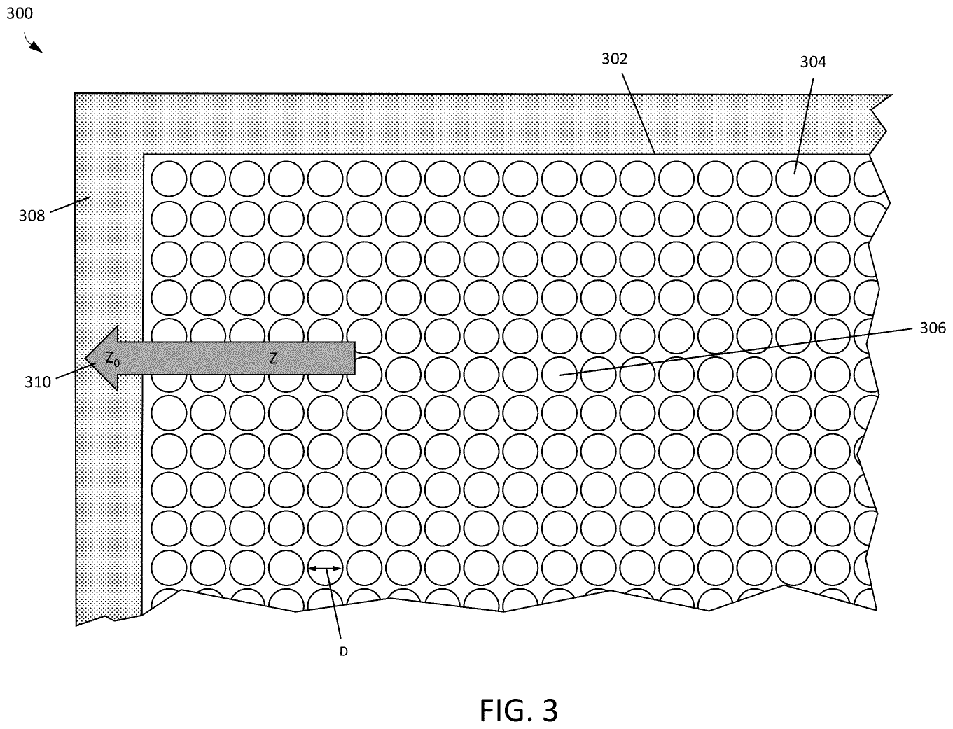

FIG. 3 illustrates one example of a real, finite 2D transducer array 300 having perimeter edges 302. The transducers (e.g., transducers 304) located closer to the perimeter edges 302, typically have different boundary conditions as compared to other transducers (e.g., transducers 306) located closer to the center of the array 300. More specifically, acoustic impedance (Z) is a physical property of a material and describes how much resistance an ultrasound beam or acoustic wave encounters as it passes through that material. In practice, the effects of acoustic impedance become noticeable at interfaces between different materials (e.g., such as the cavities of the array transducers and a solid boundary 308). More particularly, an ultrasound beam gets reflected as it travels from a material having a smaller acoustic impedance into another material having a larger acoustic impedance. As depicted in FIG. 3, an ultrasound beam (grey arrow 310) travels from the CMUT cavities 304, 306, of the array 300 into the surrounding solid boundary 308. Here, each cavity of the array has a uniform diameter, D, and the acoustic impedance difference (Z₀−Z) between the array and the boundary is large. This is because the array is formed by etching, for example, thousands of circular cavities, such that the material density of the transducer array is much smaller than that of the solid boundary material. Consequently, a large fraction of the incident ultrasound beam may be reflected back into the array and adversely impact the overall acoustic imaging quality and performance of the array.

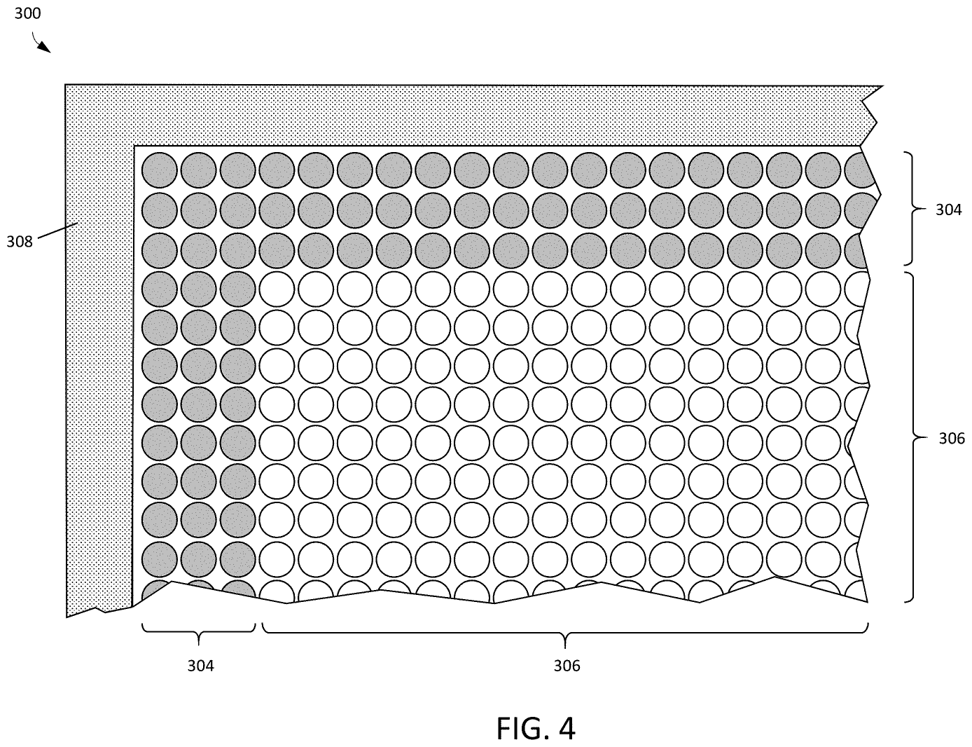

One possible way to address such boundary conditions is to design the edge transducers 304 as “dummies,” i.e., non-functional transducers during imaging, in order to improve overall array imaging quality. To illustrate, in FIG. 4 the unshaded transducers 306 are functional transducers that are used in acoustic imaging, whereas the shaded transducers 304 are non-functional transducers that do not participate in acoustic imaging and correspond to the outer 3 rows/columns of the array. However, there are at least two drawbacks associated with such an approach. First, the overall effective transducer area is reduced since the edge transducers are excluded for imaging purposes. Second, due to the acoustic impedance mismatch that still exists at the boundary of surrounding materials and the outermost row/column of transducer cavities, there is acoustic reflection at this interface. Although the edge (shaded) transducers 304 are excluded for imaging, back-reflected acoustic waves may still affect the center transducers 306 to a certain extent.

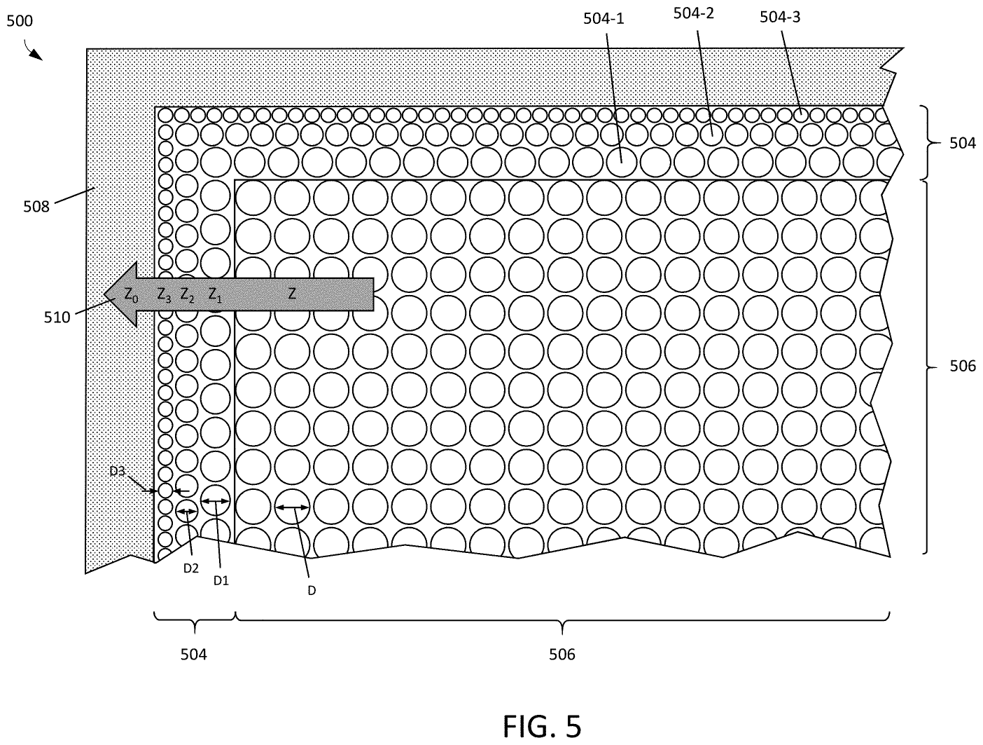

Referring now to FIG. 5, there is shown an ultrasonic transducer array 500 having a varied cavity diameter profile, in accordance with an embodiment. In the exemplary embodiment depicted, a first group of functional transducers (e.g., MUTs or more specifically CMUTs) is disposed generally in an inner region 506 of the array, with each transducer of the inner region 506 having a first diameter, D. In addition, there are a plurality of edge transducers in an outer region 504 having a varying (in this example tapered) cavity profile in an outward direction from the center of the array. More specifically, the exemplary embodiment depicts three sets of edge transducers (504-1, 504-2, 504-3) with successively smaller cavity diameters (D₁, D₂, D₃, respectively) in a direction toward the solid boundary 508. As illustrated by the grey arrow 510 in FIG. 5, the gradual tapering of cavity diameters in turn translates into a gradual (instead of abrupt) increase of acoustic impedances, particularly, Z₃>Z₂>Z₁>Z. In one example, the degree of cavity diameter tapering (and thus the degree of acoustic impedance increase may be linear or, alternatively, a non-linear decay, such as exponential for example). By way of more specific examples, the cavity diameters may decrease at a linear rate of about 20%, 40%, 60%, respectively, with each set. Again, it should be appreciated that other values are also contemplated. In any case, the acoustic impedance difference between adjacent cavities of transducers in the outer region 504 is relatively smaller due to the gradual change in cavity diameters. Although it is still the case that Z₀>Z₃ at the array boundary, this difference (Z₀−Z₃) is significantly reduced as compared to (Z₀−Z). Moreover, at each impedance interface, the impedance difference may be made small, including (Z₁−Z), (Z₂−Z₁), (Z₃−Z₂), and (Z₀−Z₃). Collectively, this arrangement may result in a very small amount of the initial ultrasound beam or acoustic wave being reflected back into the transducer array, thereby helping to maintain the imaging quality.

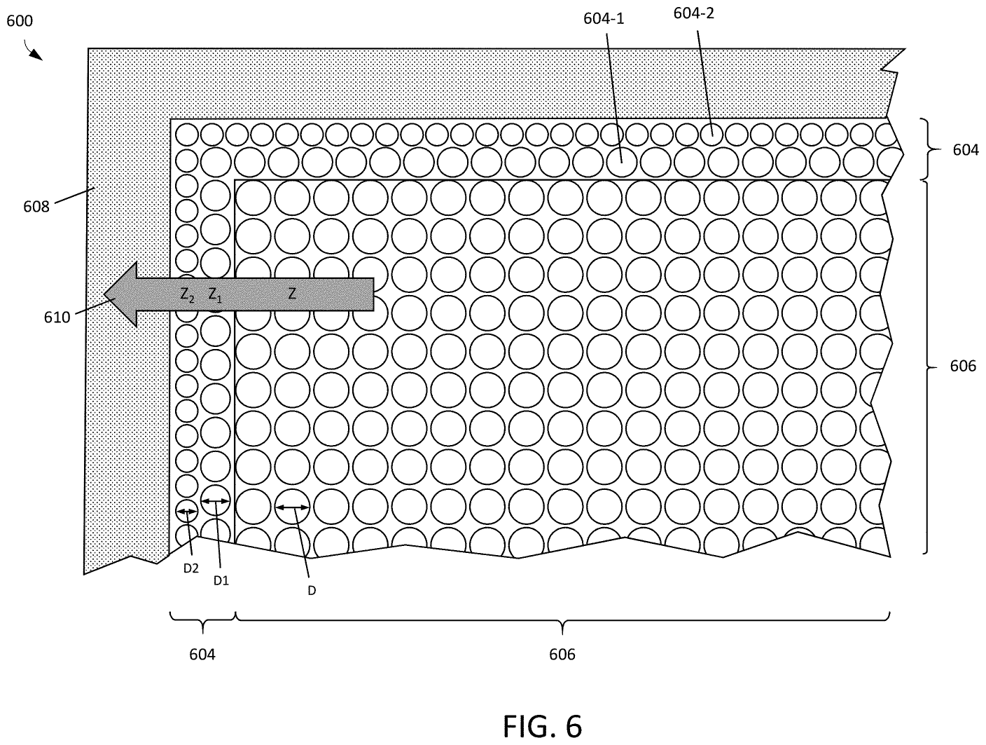

It will be appreciated that a different number of edge transducer sets may be used. For example, FIG. 6 shows an ultrasonic transducer array 600 having another varying (e.g., tapered) cavity diameter profile, in accordance with an embodiment. In the exemplary embodiment depicted, a first group of functional transducers is disposed generally in an inner region 606 of the array, with each transducer of the inner region 606 having a first diameter, D. In addition, there are a plurality of edge transducers in an outer region 604 having a tapered cavity profile in an outward direction from the center of the array. More specifically, the exemplary embodiment depicts two sets of edge transducers (604-1, 604-2) with successively smaller cavity diameters (D₁, D₂, respectively) in a direction toward the solid boundary 608. As illustrated by the grey arrow 610 in FIG. 6, the gradual tapering of cavity diameters in turn translates into a gradual (instead of abrupt) increase of acoustic impedances, particularly, Z₂>Z₁>Z. Still other embodiments may utilize a larger number of non-functional edge transducer sets (e.g., 4, 5, 6 or more), so long as the desired acoustic reflection reduction is achieved while also maintaining a sufficiently sized functional transducer array.

From a processing standpoint, because the transducer array cavities are formed by etching (e.g., a CMUT cavity oxide layer), a CMUT cavity photolithography patterning mask may be modified to form the tapered cavity diameter features at the transducer array edges. It will be appreciated that the above described embodiments provide an advantageous solution to address and resolve transducer array edge effect issues. Properly engineered tapered cavities at the transducer array edges may not only enhance imaging quality by reducing the reflection of acoustic waves at the array boundary, but also can increase chip area usage by reducing the overall dummy transducer area with respect to the entire transducer array. That is, the use of reduced cavity diameter, non-functional transducers leaves more room for functional transducers on the chip real estate.

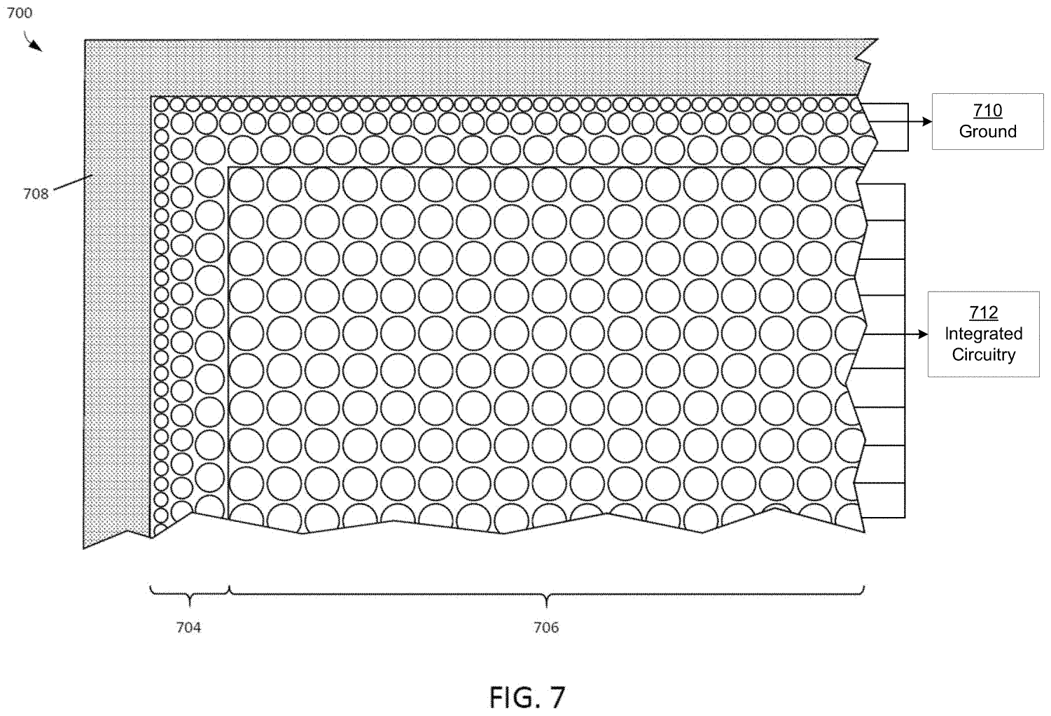

As described above, edge transducers may be made non-functional in various ways. One approach to forming the edge transducers as non-functional transducers is shown in FIG. 7. The illustrated transducer array 700 includes edge transducers 704 and central transducers 706. The edge transducers 704 and central transducers 706 both include transducer bottom electrodes, not visible in the top view of FIG. 7 For example, the edge transducers 704 and central transducers 706 may include the transducer bottom electrodes 112 of FIG. 1, or any other suitable transducer bottom electrodes. The transducer bottom electrodes of the edge transducers 704 may be (permanently, e.g., through a bypass line) electrically connected to ground 710, in some embodiments, rendering them non-functional. In contrast, the transducer bottom electrodes of the central transducers 706 may be controlled with a suitable voltage signal to make them functional, for example by electrically connecting the transducer bottom electrodes of the central transducers 706 to integrated circuitry 712. The integrated circuitry 712 may control the central transducers 706 by applying signals to the transducer bottom electrodes and/or by receiving electrical signals from the transducer bottom electrodes of the central transducers 706. In this manner, the central electrodes 706 may be controlled to perform acoustic imaging.

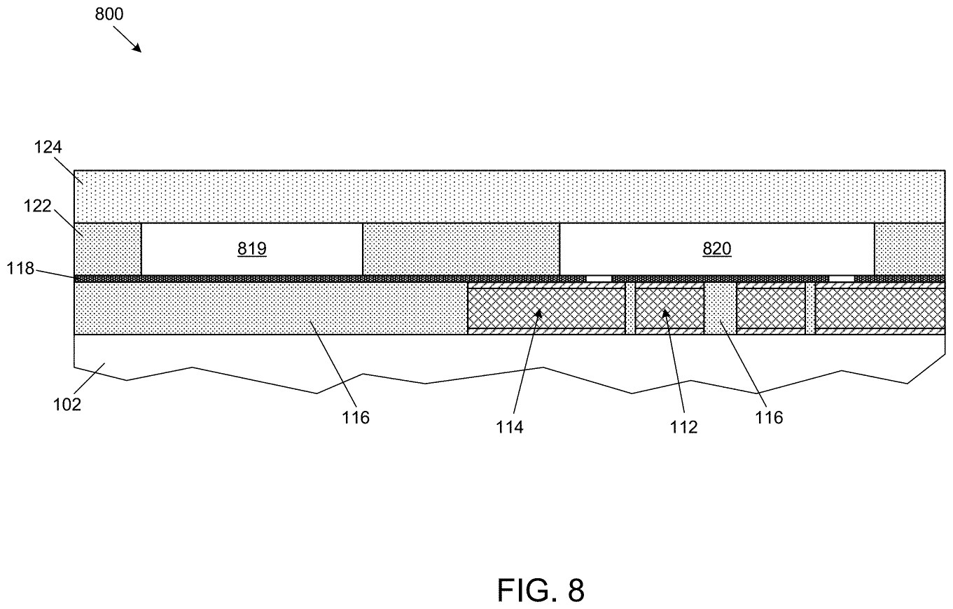

Alternatively, another manner in which the edge transducers may be made non-functional in by forming them without transducer bottom electrodes. An exemplary cross-sectional view of such a transducer assembly 800 is shown in FIG. 8. The transducer assembly 800 includes a substrate 102, an insulation layer 116, and a transducer bottom electrode 112 and bypass metal structures 114 formed in insulation layer 116 (e.g., as described in connection with FIG. 1 herein). A bottom cavity layer 118 is formed over the insulation layer 116, transducer bottom electrode 112, and bypass metal structures 114, and a membrane support layer 122 is formed over the bottom cavity layer 118. A transducer membrane 124 seals cavities 819 and 820.

In some embodiments, a non-functional transducer (e.g., transducers 504 or 604) is formed in the membrane support layer 122 by cavity 819. Cavity 819 is not formed over or connected to a transducer bottom electrode 112 or bypass metal structures 114. Cavity 819 is disposed over only the insulation layer 116. In this manner, the non-functional transducer is not electrically active or configured to perform acoustic imaging. A functional transducer (e.g., transducers 506 or 606) is formed in the membrane support layer 122 by cavity 820 which is associated with transducer bottom electrode 112 and bypass metal structures 114, causing the functional transducer to be electrically active and/or configured to perform acoustic imaging.

According to some embodiments described herein, the non-functional MUTs are disposed only around a perimeter of an array of active MUTs. For example, referring to FIG. 5-7, the edge transducers are located only around the perimeter of the array of central transducers. There are no non-functional transducers interspersed in the array of central transducers in those non-limiting examples.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, some aspects of the technology may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

What is claimed is:

1. An ultrasonic transducer array, comprising:
a plurality of functional micromachined ultrasonic transducers (MUTs), each having a cavity of a first diameter; and
one or more groups of non-functional MUTs disposed about a perimeter of the functional MUTs, the one or more groups of non-functional MUTs having a cavity of a second diameter that is smaller than the first diameter.

2. The ultrasonic transducer array of claim 1, wherein the one or more groups of non-functional MUTs have successively smaller cavity diameters in an outward direction with respect to the array.

3. The ultrasonic transducer array of claim 2 wherein the successively smaller cavity diameters decrease in a linear fashion in the outward direction.

4. The ultrasonic transducer array of claim 2 wherein the successively smaller cavity diameters decrease in a non-linear fashion in the outward direction.

5. The ultrasonic transducer array of claim 4, wherein the successively smaller cavity diameters decrease in an exponential fashion in the outward direction.

6. The ultrasonic transducer array of claim 3, further comprising at least three groups of non-functional MUTs having successively smaller cavity diameters in the outward direction with respect to the array.

7. The ultrasonic transducer array of claim 1, wherein the functional MUTs are configured for use in acoustic imaging, and the non-functional MUTs are not configured for use in acoustic imaging.

8. The ultrasonic transducer array of claim 7, wherein the non-functional MUTs are formed without functional electrodes.

9. An ultrasonic transducer device, comprising:
first micromachined ultrasonic transducers (MUTs) arranged in an array on a substrate, the first MUTs each comprising a first cavity of a first diameter such that the ultrasonic transducer device comprises first cavities of the first diameter; and
second cavities of a second diameter disposed about a perimeter of the array of first MUTs, the second diameter different than the first diameter,
wherein:
the ultrasonic transducer device lacks transducer bottom electrodes associated with the second cavities; or
the ultrasonic transducer device includes transducer bottom electrodes associated with respective cavities of the second cavities that are permanently electrically grounded.

10. The ultrasonic transducer device of claim 9, further comprising electrodes disposed between the first cavities and the substrate and no electrodes disposed between the second cavities and the substrate.

11. The ultrasonic transducer device of claim 9, further comprising:
first electrodes disposed between the first cavities and the substrate and coupled to ultrasonic transducer integrated circuitry; and
second electrodes disposed between the second cavities and the substrate and permanently coupled to ground.

12. The ultrasonic transducer device of claim 9, wherein the second cavities are arranged in successive rings disposed about the perimeter of the array, and
the second cavities disposed in rings further from the perimeter of the array have successively smaller second diameters than second cavities disposed in rings closer to the perimeter of the array.

13. The ultrasonic transducer device of claim 12, wherein the second diameters successively decrease in a linear fashion from an innermost ring adjacent the perimeter of the array to an outermost ring furthest from the perimeter of the array.

14. The ultrasonic transducer device of claim 12, wherein the second diameters successively decrease in a non-linear fashion from an innermost ring adjacent the perimeter of the array to an outermost ring furthest from the perimeter of the array.

15. The ultrasonic transducer device of claim 14, wherein the second diameters successively decrease in an exponential fashion from an innermost ring adjacent the perimeter of the array to an outermost ring furthest from the perimeter of the array.

16. The ultrasonic transducer device of claim 12, wherein the second cavities are arranged in at least three successive rings disposed about the perimeter of the array.

17. The ultrasonic transducer device of claim 9, wherein the first MUTs are configured to be electrically active during acoustic imaging, and the second cavities are configured to be electrically inactive during the acoustic imaging.

18. A method of manufacturing an ultrasonic transducer device, the method comprising:
forming an array of first micromachined ultrasonic transducers (MUTs) on a substrate by forming first cavities having a first diameter;
forming second cavities disposed about a perimeter of the array, the second cavities having a second diameter different than the first diameter,
wherein the first MUTs are configured for use in acoustic imaging, and
the second cavities are configured to be electrically inactive during acoustic imaging.

19. The method of claim 18, wherein forming the second cavities comprises forming the second cavities in successive rings disposed about the perimeter of the array, wherein
the second cavities in rings further from the perimeter of the array have successively smaller second diameters than second cavities in rings closer to the perimeter of the array.
